(19) United States
(12) Patent Application Publication
(10) Pub. No.: US 2023/0013736 A1
(43) Pub. Date: Jan. 19, 2023

(54) PROCESS FOR OBTAINING A PRE-VASCULARIZED DERMAL-EPIDERMAL TISSUE

(71) Applicants: ADHARA, Paris (FR); CENTRE D'ETUDE DES CELLULES SOUCHES (CECS), Corbeil Essonnes (FR); UNIVERSITE D'EVRY VAL D'ESSONNE, Evry (FR)

(72) Inventors: Christine BALDESHI, Villemoisson Sur Orge (FR); Sophie DOMINGUES, Corbeil-Essones (FR); Christelle LAURENSOU, Dijon (FR); Marielle BOUSCHBACHER, Chambolle-Musigny (FR); Sophie CLEMENT, Corcelles-les-Monts (FR); Guillaume OLIVE, Fontaine-les-Dijon (FR)

(21) Appl. No.: 17/905,290

(22) PCT Filed: Mar. 2, 2021
(86) PCT No.: PCT/FR2021/050353

§ 371 Date: Aug. 30, 2022

(30) Foreign Application Priority Data
Mar. 2, 2020 (EP) 20305213.9

(51) Int. Cl. A61L 27/60; A61L 27/22; A61L 27/38

(52) U.S. Cl. CPC A61L 27/60; A61L 27/225; A61L 27/3804; A61L 27/3808; A61L 27/3813; A61L 27/3834; A61L 27/3891; A61L 27/3895

(57) ABSTRACT

The invention relates to a process for obtaining a skin substitute, comprising the following steps:
a) mixing fibroblasts, endothelial cells and hydrogel of exclusively biological origin;
b) incubating the mixture obtained in step a) for a sufficient time and under suitable conditions to obtain a pre-vascularized dermis;
c) adding keratinocytes to the pre-vascularized dermis of step b) to obtain a skin substitute; wherein said fibroblasts, endothelial cells and keratinocytes were obtained from pluripotent stem cells.

The present invention relates to a process for obtaining a pre-vascularized dermal-epidermal tissue from keratinocytes, from fibroblasts and from endothelial cells derived from embryonic stem cells or induced pluripotent stem cells, in a matrix of hydrogel of exclusively biological origin.

### CONTEXT OF THE INVENTION

Skin substitutes are artificial tissues created in a laboratory, composed of a cellularized or noncellularized matrix. These skin substitutes are capable of replacing a part of the skin that is absent or defective in patients suffering from burns, chronic wounds, etc.; which constitutes an essential alternative when “standard” treatments are a failure. The current standard treatment for treating these wounds is an autologous skin graft, which, while being capable of covering the lack of tissue and restoring the barrier function using the skin tissue of the patient, has the drawback that the site of the wound experiences considerable contraction and random remodeling of the tissues.

The availability of autologous skin is also a limitation in cases where a patient's injuries exceed more than 60% of his or her total body surface; in these cases, the injuries may not be adequately covered by autografts because of the lack of sampleable tissues. The treatment therefore requires the use of alternative strategies, usually cadaver allografts. The latter act mainly as a temporary dressing for protecting and simulating healing in the bed of the wound before the placing of an autograft.

In the case of serious burns, necrotizing infections of soft tissues, after a trauma, or a tumor resection, the amount of tissue sampled is considerable. In clinical practice, such samplings are generally treated with double-thickness autologous skin grafts. However, because of the restrictive healing of these grafts and the limited donor site capacity in seriously burned patients, there is a high demand for alternative therapeutic approaches. Skin tissue engineering is a rapidly growing field in plastic and reconstructive surgery. It concentrates on the implantation of artificial skin substitutes, which should promote the rapid healing of wounds and prevent loss of liquid and infections. Furthermore, the skin substitutes should avoid significant scars and contractions.

There are many skin substitutes on the market: Dermagraft® (cellular dermal substitute based on collagen, elastin and allogenic fibroblasts), MatriDerm® (freeze-dried acellular dermal substitute based on non-crosslinked elastin and collagen), Apligraf® (dermal-epidermal substitute based on collagen with human primary keratinocytes and fibroblasts), etc. However, all these dressings have several drawbacks, including the absence of cells which allow rapid revascularization of the affected areas. Indeed, the clinical transition of skin substitutes has been limited by the insufficient diffusion of oxygen and nutrients in the graft. At diffusion distances of greater than 150-200 μm, perfusable vascular network-free tissue constructs undergo rapid cell death and necrosis. Consequently, in order to obtain a successful implantation, the artificial replacement requires rapid perfusion and integration with the recipient vascular system.

Numerous research teams have shown that the addition of endothelial cells to tissues generated in vitro (skin or other tissue/organs) makes it possible to improve the taking of the graft and to limit necrosis. The presence of these endothelial cells allows rapid perfusion of the grafted tissue by anastomosis with the host's tissues.

International application WO2016/209166 describes a method for obtaining a layer of vascularized dermis by combining the following cell types, all derived from pluripotent stem cells:

endothelial cells, vascular smooth muscle cells/pericytes, and optionally fibroblasts.

In particular, international application WO2016/209166 teaches that it is necessary to combine smooth muscle cells/pericytes with endothelial cells in order to obtain functional and lasting vascularization.

Surprisingly, the inventors have demonstrated that it is possible to obtain a pre-vascularized dermis, and a pre-vascularized dermal-epidermal tissue, from endothelial cells and fibroblasts derived from pluripotent stem cells, in the absence of vascular smooth muscle cells/pericytes.

### SUMMARY OF THE INVENTION

The present invention describes a process for preparing pre-vascularized dermal-epidermal tissues from cells derived from pluripotent stem cells, preferably from cells derived from human pluripotent stem cells (hPSCs) in a matrix of hydrogel of exclusively biological origin.

More particularly, the invention relates to a process for obtaining a skin substitute, comprising the following steps:
a) mixing fibroblasts, endothelial cells and hydrogel of exclusively biological origin;
b) incubating the mixture obtained in step a) for a sufficient time and under suitable conditions to obtain a tissue of “pre-vascularized dermis” type;
c) adding keratinocytes to the pre-vascularized dermis of step b) to obtain a skin substitute;
d) optionally, incubating at the air-liquid interface for a sufficient time to obtain a pluristratified pre-vascularized dermal-epidermal tissue;

wherein said fibroblasts, endothelial cells and keratinocytes were obtained from pluripotent stem cells.

According to one particularly preferred embodiment, said fibroblasts, endothelial cells and keratinocytes were obtained from human pluripotent stem cells.

According to one particularly preferred embodiment, no vascular smooth muscle cell (or pericyte) is added to the mixture of step a).

According to the invention, the incubation of step b) takes place for a sufficient time and under suitable conditions to obtain a pre-vascularized dermis.

The term “pre-vascularized dermis” or “pre-vascularized dermis equivalent” or “dermis substitute” is intended to mean a tissue comprising fibroblasts and endothelial cells included in a matrix.

The term “pre-vascularized dermal-epidermal tissue”, “dermal-epidermal tissue” or “skin substitute” is intended to mean a tissue comprising a pre-vascularized dermis on which a monolayer of keratinocytes lies.

The term “pluristratified pre-vascularized dermal-epidermal tissue” is intended to mean a tissue comprising a pre-vascularized dermis on which lies a pluristratified epidermis comprising several layers of differentiated keratinocytes.

The term “hydrogel of exclusively biological origin” is intended to mean any hydrogel comprising proteins of animal or plant origin, such as chitosan, collagen, elastin, gelatin, fibrin, or glycosaminoglycans. In the context of the present invention, the preferred hydrogel is human plasma fibrin.

According to the present invention, the hydrogel does not contain Poly Ethylene Glycol (PEG) or other synthetic polymers commonly used in tissue substitute matrices.

In one embodiment, step b) takes place for at least 4 days, preferably at least 6 days, even more preferably at least 7 days.

In one embodiment, step b) takes place for at least 14 days, preferably at most 12 days, even more preferably at most 10 days, 9 days, 8 days or 7 days.

In one preferred embodiment of the invention, step b) takes place for approximately 7 days.

According to one embodiment of the invention, the culture medium of step b) is composed of growth factors conducive to the proliferation and maturation of fibroblasts and of endothelial cells. The term “maturation” is understood to mean, for fibroblasts: the production of extracellular matrix and the remodeling of the matrix; and for endothelial cells, the remodeling of the matrix and the assembly of a primary vascular network.

According to one embodiment of the invention, the culture medium of step b) comprises vascular endothelial growth factor (VEGF), preferably stabilized VEGF. Preferably, the VEGF or stabilized VEGF is present in the culture medium of step b) at a concentration of from 5 to 60 ng/mL, preferably 45 to 55 ng/mL, even more preferably approximately 50 ng/mL.

According to one embodiment of the invention, the culture medium of step c) comprises growth factors conducive to the proliferation of keratinocytes and to their optional subsequent stratification and to the stabilization of the primary vascular network created during step b).

According to one embodiment of the invention, the culture medium of step c) comprises VEGF, preferably stabilized VEGF. Preferably, the VEGF or stabilized VEGF is present in the culture medium of step c) at a concentration of from 5 to 60 ng/mL, preferably 45 to 55 ng/mL, even more preferably approximately 50 ng/mL.

Advantageously, the various cell types used (fibroblasts, endothelial cells, keratinocytes) are obtained by differentiation of pluripotent stem cells, preferably human induced pluripotent stem cells.

In one embodiment, the invention is not based on the use of cells requiring the destruction of an embryo.

Those skilled in the art have, at their disposal, several methods for obtaining homogeneous differentiated cell populations from human pluripotent stem cells.

Many processes for differentiating human pluripotent stem cells into fibroblasts, endothelial cells or keratinocytes have been described in the literature.

Typically, processes for differentiating stem cells into fibroblasts are described in Shamis Y, Silva E A, Hewitt K J, Brudno Y, Levenberg S, Mooney D J, et al. (2013) Fibroblasts Derived from Human Pluripotent Stem Cells Activate Angiogenic Responses in Vitro and In Vivo, PLoS ONE 8(12): e83755.

Typically, processes for differentiating stem cells into endothelial cells have been described in applications EP3327118, WO2018101466 and US20180023051.

Typically, a process for differentiating IPS cells into keratinocytes is described in international application WO2009/156398.

The present invention also relates to the various therapeutic and nontherapeutic uses of said skin substitutes.

In one aspect, the invention relates to a skin substitute as described above, as a medicament.

In one aspect, the invention relates to a skin substitute as described above, for use in a treatment method, such as the treatment of burns and chronic skin wounds.

In another aspect, the invention relates to the use of a skin substitute as described above, in a method for screening for compounds and molecules intended to be in contact with the skin and notably for cosmetic or therapeutic purposes.

In another aspect, the present invention relates to a population of skin substitutes obtained according to the process described above. Advantageously, this population of skin substitutes represents a homogeneous, reproducible collection of samples, obtained under clinical conditions and in a large amount.

### BRIEF DESCRIPTION OF THE FIGURES

Other characteristics, details and advantages of the invention will emerge from reading the appended figures.

FIG. 1 represents the process for obtaining dermis equivalent from endothelial cells, from fibroblasts and from fibrin.

FIG. 2 represents an example of the process for obtaining dermal-epidermal tissue according to the invention.

FIG. 3 represents an example of dermal-epidermal tissue according to the invention sutured onto a mouse.

FIG. 4 represents the formation of circular structures composed of endothelial cells in a dermal-epidermal tissue obtained according to the process of the invention in which the biomaterial is fibrin and the fibroblasts and endothelial cells derived from human iPSCs are seeded in a 1:40 ratio (EC:CNT medium).

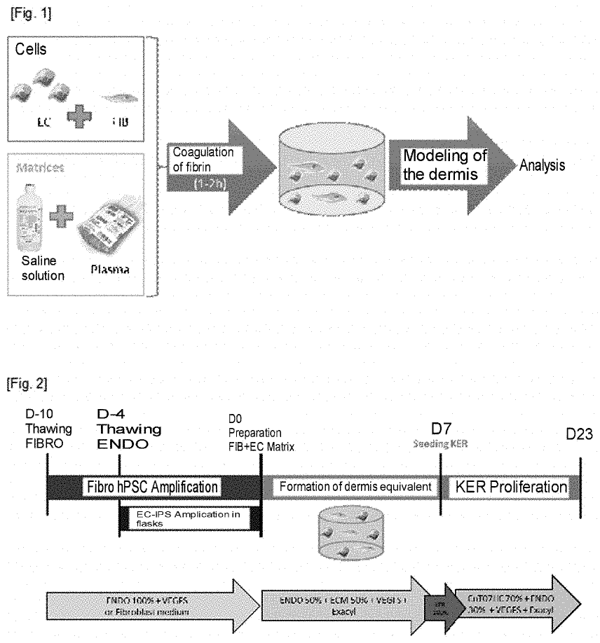

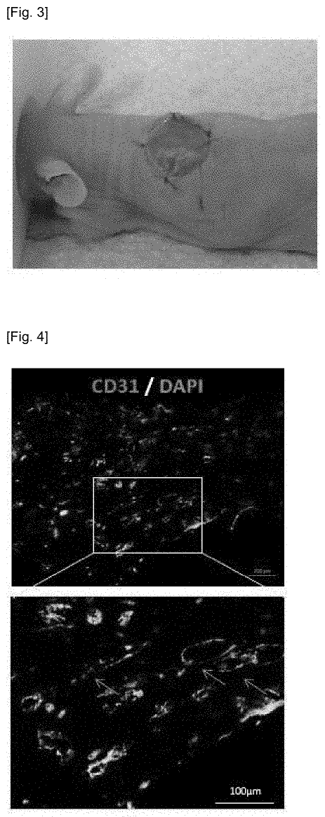

### EXAMPLES

Example 1: Obtaining of Fibroblasts, of Endothelial Cells and of Keratinocytes from Induced Pluripotent Stem Cells

A human line of induced pluripotent stem cells was used to obtain, via 3 suitable differentiation processes, fibroblasts (FIB), endothelial cells (EC) and keratinocytes (KER).

### Example 2: Obtaining of a Pre-Vascularized Dermis

The fibroblasts (FIB) and the endothelial cells (EC): the FIB and the EC were preamplified upstream of the preparation of the tissues in culture flasks and in F-CnT medium (CelInTec) for the FIB and in StemPro34 (Invitrogen) or ENDO-CnT (CelInTec) medium supplemented with VEGF at 50ng/mL for the EC. When the 2 cell types were ready, they were passaged and counted. The cells at the ratio of 1 FIB for 40 EC were mixed with fibrin. The fibrin is obtained by mixing plasma and saline solution under the conditions described in Table 1.

TABLE 1

| Fibrin components | Initial concentrations | Volume for 5 mL |
|---|---|---|
| Human plasma | — | 2 mL |
| NaCl | 0.9% | 2.6 mL |
| Exacyl ® | 0.1 g/mL | 20 μL |
| CaCl₂ | 0.1M | 400 μL |

The “cells+fibrin” solution was deposited in inserts of 1 cm², i.e. 600 μL/insert, with 10 000 FIB+400 000 EC. The plates were then placed at +37° C. to allow coagulation of the matrix for a minimum of 1 h. When the fibrins had set, medium was added: 1 mL in the inserts and 2 mL in the wells so that the tissue is completely immersed. The medium used is a ⅓ mixture of F-CnT medium and ⅔ of “EC” medium for 1 day. The following day (D1) and up to D7, the medium was changed every 2 days for the medium composed of ⅓ of F-CnT medium and ⅔ of EC medium, with the same volumes as mentioned.

### Example 3: Obtaining of a Pre-Vascularized Dermal-Epidermal Tissue

The culture was carried out across approximately 14 days in an incubator regulated at +37° C. with 5% CO₂ for the dermal tissues and 21 days for the dermal-epidermal tissues. The F-CnT medium of the mixture was replaced with the ECM medium on D1 to allow the FIB to secrete extracellular matrix and stabilized VEGF at 50 ng/mL.

On D7, a part of the tissues was left under the same conditions up to D14 in order to perform an analysis of the dermis alone, and the other part was seeded with keratinocytes (KER) on the matrices in CnT07.HC medium with 1.6 mg/mL of Exacyl® for 24-48 h at 100 000/cm². On D9, the tissues with KER were placed at the air-liquid interface to allow stratification of the epidermis. The medium used was then composed of Airlift medium at 70%, ENDO-CnT medium at 30% with Exacyl® at 1.6 mg/mL and stabilized VEGF at 50 ng/mL.

These experiments made it possible to obtain a pre-vascularized dermal-epidermal tissue which, once grafted onto the mouse, did not exhibit any necrosis.

1. A process for obtaining a skin substitute, comprising the following steps:
a) mixing fibroblasts, endothelial cells and hydrogel of exclusively biological origin;
b) incubating the mixture obtained in step a) in a culture medium for a sufficient time and under suitable conditions to obtain a pre-vascularized dermis tissue;
c) adding keratinocytes to the pre-vascularized dermis tissue of step b) to obtain a skin substitute; and
d) optionally, incubating the skin substitute at an air-liquid interface for a sufficient time to obtain a pluristratified pre-vascularized dermal-epidermal tissue,
wherein said fibroblasts, endothelial cells and keratinocytes were obtained from pluripotent stem cells.

2. The process of claim 1, wherein no vascular smooth muscle cell is added to the mixture of step a).

3. The process of claim 1, wherein the step of incubating takes place for 7 days.

4. The process of claim 1, wherein vascular epidermal growth factor (VEGF), preferably stabilized VEGF, is present in the culture medium of step b), preferably at a concentration from 30 to 60 ng/mL, preferably 45 to 55 ng/mL.

5. The process of claim 1, wherein VEGF, is present in the culture medium of step c).

6. The process of claim 1, wherein the fibroblasts and the endothelial cells of step a) are obtained from human induced pluripotent stem cells.

7. The process as of claim 1, wherein the keratinocytes of step c) are obtained from human induced pluripotent stem cells.

8. A skin substitute comprising a pre-vascularized dermis and a monolayer of keratinocytes obtained according to the process of claim 1.

9. A skin substitute comprising a pre-vascularized dermis and a pluristratified epidermis, obtained according to the process of of claim 1.

10. A method of treating a wound in a subject in need thereof, comprising grafting the skin substitute according to claim 8 onto the wound.

11. A method of treating a wound in a subject in need thereof, comprising grafting the skin substitute according to claim 9 onto the wound.

12. A method for screening for compounds and molecules intended to be in contact with the skin for cosmetic and/or therapeutic purposes, comprising the step of contacting the skin substitute as claimed in claim 8 with the compounds and/or molecules.

13. A population of skin substitutes obtained according to the process as claimed in claim 7.

14. A method for screening for compounds and/or molecules intended to be in contact with the skin and notably for cosmetic and/or therapeutic purposes, comprising the step of
contacting the skin substitute as claimed in claim 9 with the compounds and/or molecules.

15. The process of claim 1, wherein stabilized vascular epidermal growth factor (VEGF), is present in the culture medium of step b).

16. The process of claim 1, wherein stabilized vascular epidermal growth factor (VEGF), is present in the culture medium of step b) at a concentration from 30 to 60 ng/mL.

17. The process of claim 1, wherein stabilized vascular epidermal growth factor (VEGF), is present in the culture medium of step b) at a concentration from 45 to 55 ng/mL.

18. The process of claim 1, wherein stabilized vascular epidermal growth factor (VEGF), is present in the culture medium of step c).

19. The process of claim 1, wherein stabilized vascular epidermal growth factor (VEGF), is present in the culture medium of step c) at a concentration from 30 to 60 ng/mL.

20. The process of claim 1, wherein stabilized vascular epidermal growth factor (VEGF), is present in the culture medium of step c) at a concentration from 45 to 55 ng/mL.
